(19) United States
(12) Patent Application Publication
(10) Pub. No.: US 2026/0048023 A1
(43) Pub. Date: Feb. 19, 2026

(54) LIPID NANOPARTICLE COMPRISING A DNA-BINDING PROTEIN

(71) Applicant: SEQIRUS INC., Holly Springs, NC (US)

(72) Inventor: Steven ROCKMAN, Holly Springs, NC (US)

(21) Appl. No.: 19/104,432

(22) PCT Filed: Aug. 18, 2023
(86) PCT No.: PCT/IB2023/058263

§ 371 Date: Feb. 18, 2025

Related U.S. Application Data
(60) Provisional application No. 63/371,914, filed on Aug. 19, 2022.

(51) Int. Cl. A61K 9/51; A61K 31/711; A61K 47/24; A61K 47/64

(52) U.S. Cl. CPC A61K 9/5169; A61K 9/5123; A61K 31/711; A61K 47/24; A61K 47/64

(57) ABSTRACT

The present disclosure relates to lipid nanoparticles for delivery of DNA, the lipid nanoparticle comprising therein a DNA-binding protein or peptide bound to the DNA, and uses thereof.

### RELATED APPLICATION DATA

The present application claims priority from US Patent Application No. 63/371,914 filed 19 Aug. 2022 entitled “Lipid nanoparticle comprising a DNA-binding protein”, the entire contents of which is hereby incorporated by reference.

### FIELD

The present disclosure relates to lipid nanoparticles for delivery of DNA, the lipid nanoparticle comprising therein a DNA-binding protein or peptide bound to the DNA, and uses thereof.

### BACKGROUND

Nucleic acid therapeutics, including DNA vaccines and gene therapy, are promising approaches for the treatment and prevention of various diseases by targeting their genetic ‘blueprint’ in vivo. The clinical translation of DNA-based therapeutics relies upon the use of delivery vehicles that improve stability, facilitate internalization of the DNA to the nucleus and increase target affinity. Efficient DNA delivery requires transfer across both the cytoplasm and the nuclear membrane to the nucleus. This is a major limitation of current DNA therapeutic and gene therapy approaches.

Existing non-viral delivery systems, such as lipid nanoparticles that effectively deliver RNA do not work as well for the delivery of DNA due to the larger size of the molecule being delivered and the need to cross the nuclear envelope. Historically, delivery of DNA has required the use of viral vectors or physical DNA delivery by electroporation or a needle-free injection systems. However, these methods only permit small lengths of linear DNA (˜200-300 base pairs) to be delivered to the nucleus.

In addition to the physical barriers of DNA delivery, current non-viral lipid delivery vehicles are formed from cationic lipids and other ionisable lipid components such as neutral lipids, cholesterol and PEGylated lipids. Cationic lipids are amphiphilic molecules having a lipophilic region containing one or more hydrocarbon groups and a hydrophilic region containing at least one positively charged polar head group. Cationic lipids and nucleic acids form a positively charged complex, making it easier for the nucleic acids to pass through the plasma membrane of the cell.

However, several adverse cytotoxic effects of cationic lipids are known including, production of reactive oxygen species and accumulation in plasma due to poor degradation by humans. Thus, much effort has focused on identifying novel lipids or particular lipid compositions that can protect nucleic acids from degradation and elimination, as well as provide effective intracellular delivery and/or expression/transcription of DNA. In addition, these lipid-nucleic acid particles must be well tolerated and provide an appropriate therapeutic index so that treatment with an effective dose of nucleic acid must not be associated with unacceptable toxicity and/or risk to the patient.

Therefore, it will be apparent to the skilled person that there is an on-going need for improved delivery systems for delivery of nucleic acids, in particular improved delivery vehicles for delivery of DNA therapeutics and gene therapies.

### SUMMARY

In producing the present invention, the inventors identified that incorporation of a DNA-binding protein or peptide or a lipidated DNA-binding protein or peptide in a lipid nanoparticle may increase stability of the associated DNA and/or facilitate nucleation of the lipid nanoparticle and/or reduce the toxicity and/or adverse side effects of the lipid nanoparticle. In some forms of the disclosure, the inventors identified that incorporation of a DNA-binding protein with a nuclear localisation sequence will allow importation of the DNA into the nucleus. The inventors have also identified that incorporation of the DNA-binding protein or peptide into the lipid nanoparticle may be able to protect against toll-like receptor (TLR) stimulation/induction. Based on these findings, the inventors have produced reagents and methods for delivery of DNA, e.g., non-viral delivery of DNA into a cell, e.g., within a subject.

Accordingly, the present disclosure provides a lipid nanoparticle for delivery of DNA, the lipid nanoparticle comprising therein a DNA-binding protein or peptide bound to the DNA.

In one example, the DNA-binding protein or peptide is a lipidated DNA-binding protein or peptide.

Accordingly, the present disclosure provides a lipid nanoparticle for delivery of DNA, the lipid nanoparticle comprising therein a lipidated DNA-binding protein or peptide bound to the DNA.

In one example, the DNA-binding protein or peptide is lipidated prior to binding the DNA. In another example, the DNA-binding protein or peptide is lipidated after binding to the DNA.

In one example, the DNA-binding protein or peptide is lipidated with a lipid moiety selected from the group consisting of a fatty acid, an isoprenoid and combinations thereof.

In one example, the DNA-binding protein or peptide is lipidated with a fatty acid. For example, the fatty acid is a triglyceride, a phospholipid or a cholesteryl ester. In one example, the fatty acid is a triglyceride. In another example, the fatty acid is a phospholipid. In a further example, the fatty acid is a cholesteryl ester.

In one example, the DNA-binding protein or peptide is lipidated with an isoprenoid. For example, the isoprenoid is isoprene.

In one example, the DNA-binding protein or peptide is lipidated on a nucleophilic side chain, at the N-terminal end and/or at the C-terminal end.

In one example, the DNA-binding protein or peptide is lipidated on a nucleophilic side chain. For example, on a cysteine, a serine, a threonine, a tyrosine and/or a lysine amino acid residue. In one example, the nucleophilic side chain is a cysteine residue. In another example, the nucleophilic side chain is a serine residue. In a further example, the nucleophilic side chain is a threonine residue. In one example, the nucleophilic side chain is a tyrosine residue. In another example, the nucleophilic side chain is a lysine residue.

In one example, the DNA-binding protein or peptide is lipidated at the N-terminal end of the protein or peptide.

It will be apparent to the skilled person that the N-terminal end of the DNA-binding protein or peptide comprises a nuclear localisation signal(s) (or sequence) and/or a nuclear export signal.

In one example, the DNA-binding protein or peptide is lipidated at the C-terminal end of the protein or peptide.

In one example, the DNA-binding protein or peptide is lipidated by palmitoylation, myristoylation, fatty-acylation, esterification, prenylation or combinations thereof.

In one example, the DNA-binding protein or peptide is lipidated by palmitoylation. For example, N-terminal cysteine palmitoylation.

In one example, the DNA-binding protein or peptide is lipidated by myristoylation. For example, N-terminal glycine myristoylation.

In one example, the DNA-binding protein or peptide is lipidated by fatty-acylation. For example, lysine N-acylation. In another example, serine O-acylation.

In one example, the DNA-binding protein or peptide is lipidated by esterification. For example, C-terminal cholesterol esterification.

In one example, the DNA-binding protein or peptide is lipidated by prenylation. For example, the prenylation is farnesylation or geranylgeranylation. In one example, the prenylation is cysteine prenylation.

In one example, the DNA-binding protein or peptide is lipidated by N-terminal cysteine palmitoylation, N-terminal glycine myristoylation, lysine N-acylation, C-terminal cholesterol esterification, cysteine prenylation, serine O-acylation or combinations thereof.

In one example, the lipid moiety is linked to the DNA-binding protein or peptide by a thioether bond, an ester bond, a thioester bond and/or an amide bond.

In one example, the lipid moiety is linked to the DNA-binding protein or peptide by a thioether bond.

In one example, the lipid moiety is linked to the DNA-binding protein or peptide by an ester bond.

In one example, the lipid moiety is linked to the DNA-binding protein or peptide by a thioester bond.

In one example, the lipid moiety is linked to the DNA-binding protein or peptide by an amide bond.

In one example, the DNA-binding protein or peptide is lipidated using chemical or enzymatic lipidation. For example, the DNA-binding protein or peptide is lipidated using chemical lipidation. In one example, the chemical lipidation is selected from the group consisting of chemical ligation, click chemistry, expressed protein ligation and combinations thereof. In another example, the DNA-binding protein or peptide is lipidated using enzymatic lipidation. For example, the enzymatic lipidation is selected from the group consisting of Sortase-A mediated lipidation, transglutaminase mediated lipidation and combinations thereof. In one example, the enzymatic lipidation is performed in vivo or in vitro. For example, the enzymatic lipidation is performed in vivo. In another example, the enzymatic lipidation is performed in vitro.

In one example, the DNA-binding protein or peptide binds directly to the DNA. In another example, the DNA-binding protein or peptide binds the DNA prior to formulating the DNA into a lipid nanoparticle. In a further example, the DNA-binding protein or peptide binds the DNA in the lipid nanoparticle after formulating the DNA into a lipid nanoparticle, wherein the DNA-binding protein or peptide is within the lipid nanoparticle. For example, the DNA-binding protein or peptide binds the lipid nanoparticle encapsulated DNA.

In one example, the DNA-binding protein or peptide additionally binds to DNA on the surface of the lipid nanoparticle. In such a situation, DNA-binding protein or peptide will be present within the lipid nanoparticle and on the surface of the lipid nanoparticle. The DNA-binding protein or peptide on the surface of the lipid nanoparticle need not be the same as the DNA-binding protein or peptide within the lipid nanoparticle.

For example, a lipid nanoparticle can be formed with a DNA-binding protein or peptide bound to DNA therein and the formed lipid nanoparticle can then be coated with a DNA-binding protein or peptide to bind to any unencapsulated and/or partially encapsulated DNA.

In one example, the DNA-binding protein or peptide encapsulates the DNA. In another example, the DNA-binding protein or peptide binds on a nucleophilic side chain, at the N-terminal end and/or at the C-terminal end of the DNA. In one example, the DNA-binding protein or peptide binds on a nucleophilic side chain of the DNA. In another example, the DNA-binding protein or peptide binds at the 5′ end and/or at the 3′ end of the DNA. For example, at the 5′ end of the DNA. In another example, at the 3′ end of the DNA. For example, the DNA-binding protein or peptide does not encapsulate the DNA.

In one example, the DNA-binding protein or peptide:
a) reduces toxicity of the lipid nanoparticle,
b) stabilizes the DNA,
c) facilitates importation through the nuclear membrane,
d) protects the DNA from degradation,
e) facilitates nucleation of the lipid nanoparticle,
f) increases immunogenicity of the DNA, and/or
g) inhibits induction of signalling by one or more Toll-like receptors.

In one example, the DNA-binding protein or peptide reduces toxicity of the lipid nanoparticle.

In one example, the DNA-binding protein or peptide stabilizes the DNA.

In one example, the DNA-binding protein or peptide facilitates importation through the nuclear membrane.

In one example, the DNA-binding protein or peptide protects the DNA from degradation.

In one example, the DNA-binding protein or peptide facilitates nucleation of the lipid nanoparticle.

In one example, the DNA-binding protein or peptide increases immunogenicity of the DNA.

In one example, the DNA-binding protein or peptide inhibits induction of signalling by one or more Toll-like receptors. In one example, the DNA-binding protein or peptide does not inhibit induction of signalling by one or more Toll-like receptors.

The skilled person will understand that there are a set of Toll-like receptors, namely endosomal Toll-like receptors comprising TLR-3, TLR-7, TLR-8 and TLR-9 that recognise and bind nucleic acids, such as DNA. Activation of these receptors leads to production of inflammatory cytokines, as well as type I interferons (interferon type I).

In one example, the DNA-binding protein or peptide inhibits induction of signalling by one or more endosomal Toll-like receptors. For example, the DNA-binding protein or peptide inhibits induction of signalling by one or more Toll-like receptors selected from the group consisting of TLR-3, TLR-7, TLR-8 and TLR-9. In one example, the DNA-binding protein or peptide inhibits induction of signalling by TLR-3. In another example, the DNA-binding protein or peptide inhibits induction of signalling by TLR-7. In another example, the DNA-binding protein or peptide inhibits induction of signalling by TLR-9. In a further example, the DNA-binding protein or peptide inhibits induction of signalling by TLR-8.

In one example, the DNA-binding protein or peptide is two DNA-binding proteins or peptides (i.e., a first and a second DNA-binding protein or peptide) linked by a linker. For example, the first and second DNA-binding proteins or peptides are covalently linked by an amide bond. The present disclosure encompasses other forms of covalent and non-covalent linkages. For example, the DNA-binding proteins or peptides can be linked by a chemical linker.

In one example, the linker is a flexible linker, e.g., a flexible peptide linker. For example, the first DNA-binding protein or peptide is linked to the second DNA-binding protein via a flexible linker.

In one example, the linker is a peptide linker. For example, the first DNA-binding protein or peptide is linked to the second DNA-binding protein or peptide via a linker wherein the linker is a peptide linker comprising between 2 and 31 amino acids in length. In one example, the linker comprises the sequence (Gly₄Ser)ₙ, wherein n is between 1 and 6. For example, the linker comprises the sequence SGGGGS (GS6) or the sequence SGGGGSGGGGSGGGGSGGGGSGGGGSGGGGS (GS31). In another example, the linker comprises the sequence (Ala)ₙ, wherein n is between 2 and 31.

In one example, the linker is a rigid linker. For example, the rigid linker comprises the sequence (EAAAK)ₙ, where n is between 1 and 3. In one example, the rigid linker comprises the (EAAAK)ₙ, where n is between 1 and 10 or between about 1 and 100. For example, n is at least 1, or at least 2, or at least 3, or at least 4, or at least 5, or at least 6, or at least 7, or at least 8, or at least 9, or at least 10. In one example, n is less than 100. For example, n is less than 90, or less than about 80, or less than about 60, or less than about 50, or less than about 40, or less than about 30, or less than about 20, or less than about 10.

In one example, the DNA-binding protein or peptide is a viral or non-viral DNA-binding protein or peptide.

In one example, the DNA-binding protein or peptide is a viral DNA-binding protein. For example, the viral DNA-binding protein or peptide is from a class I, class II, and/or class VII virus. In one example, the viral DNA-binding protein or peptide is from a class I virus. In another example, the viral DNA-binding protein or peptide is from a class II virus. In a further example, the viral DNA-binding protein or peptide is from a class VII virus.

In one example, the viral DNA-binding protein or peptide is from a respiratory virus selected from the group consisting of an influenza virus, a respiratory syncytial virus, a parainfluenza virus, a metapneumovirus, a rhinovirus, a coronavirus, an adenovirus and a bocavirus.

In one example, the viral DNA-binding protein or peptide is from an influenza virus. For example, the influenza virus is influenza A. In another example, the influenza virus is influenza B.

In one example, the viral DNA-binding protein or peptide is selected from an adenovirus, a herpesvirus, a poxvirus, an adeno-associated virus, a geminivirus, a bacteriophage, a parvovirus, a heparnavirus, a hepadnavirus, a circoviridae virus, and a papovaviridae virus.

In one example, the viral DNA-binding protein or peptide is from an adenovirus virus. For example, the adenovirus is human adenovirus. In another example, the adenovirus is avian adenovirus. In another example, the adenovirus is adenovirus type-5 (Ad5). In this example, the viral DNA-binding protein is protein VII from Ad5.

In one example, the viral DNA-binding protein or peptide is from a herpesvirus. For example, the herpesvirus is Herpes simplex virus-1 (HSV-1). In another example, the herpesvirus is HSV-2. In a further example, the herpesvirus is Varicella zoster virus (VZV). In one example, the herpesvirus is Epstein-Barr virus (EBV). In another example, the herpesvirus is Cytomegalovirus (CMV). In yet another example, the herpesvirus is Roseolovirus. In a further example, the herpesvirus is Kaposi's sarcoma-associated herpesvirus (KSHV). In yet a further example, the herpesvirus is Cercopithecine herpesvirus 1, (monkey B virus). In one example, the herpesvirus is Murid herpesvirus 68 (MHV-68).

In one example, the viral DNA-binding protein or peptide is from a poxvirus. For example, the poxvirus is smallpox virus. In another example, the poxvirus is vaccinia virus. In a further example, the poxvirus is cowpox virus. In one example, the poxvirus is monkeypox virus. In another example, the poxvirus is rabbitpox virus. In yet another example, the poxvirus is orf virus. In a further example, the poxvirus is pseudocowpox. In yet a further example, the poxvirus is bovine papular stomatitis virus. In one example, the poxvirus is tanapox virus. In a further example, the poxvirus is yaba monkey tumor virus. In one example, the poxvirus is molluscum contagiosum virus (MCV).

In one example, the viral DNA-binding protein or peptide is from an adeno-associated virus.

In one example, the viral DNA-binding protein or peptide is from a geminivirus. For example, the genimivirus is begomovirus. In another example, the geminivirus is becurtovirus. In a further example, the geminivirus is capulavirus. In one example, the geminivirus is curtovirus. In another example, the geminivirus is eragrovirus. In yet another example, the geminivirus is grablovirus. In a further example, the geminivirus is mastrevirus. In yet a further example, the geminivirus is topocuvirus. In one example, the geminivirus is turneurtovirus.

In one example, the viral DNA-binding protein or peptide is from a bacteriophage.

In one example, the viral DNA-binding protein or peptide is from a parvovirus. For example, the parvovirus is bocavirus.

In one example, the viral DNA-binding protein or peptide is from a hepadnavirus. For example, the hepadnavirus is avihepadnavirus. In another example, the hepadnavirus is orthohepadnavirus. In one example, the viral DNA-binding protein or peptide is from a hepadnavirus. For example, the hepadnavirus is Hepatitis B virus (HBV).

In one example, the viral DNA-binding protein or peptide is from a circoviridae virus. For example, the circoviridae virus is beak and feather disease virus (BFDV).

In one example, the viral DNA-binding protein or peptide is from a papovaviridae virus. For example, the papovaviridae virus is human papillomavirus (HPV). In another example, the human papillomavirus is HPV16.

In one example, the viral DNA-binding protein or peptide is a nucleoprotein, a non-structural protein, a matrix protein and/or a nucleocapsid protein. For example, the viral DNA-binding protein or peptide is a nucleoprotein. In another example, the viral DNA-binding protein or peptide is a matrix protein. In a further example, the viral DNA-binding protein or peptide is a nucleocapsid protein. In another example, the viral DNA-binding protein or peptide is a non-structural protein.

In one example, the viral DNA-binding protein or peptide is a non-structural (NS) protein from a herpesvirus. For example, the viral DNA-binding protein or peptide is HSV-1. In one example, the HSV-1 DNA binding domain is a full length binding domain. In another example, the HSV-1 DNA binding domain is a truncated binding domain. In a further example, the HSV-1 DNA binding domain is a modified binding domain. For example, the 3′ end of a first HSV-1 DNA binding domain is linked to the 5′ end of a second HSV-1 DNA binding domain.

In one example, the viral DNA-binding protein or peptide is a capsid protein from a beak and feather disease virus. In one example, the viral DNA-binding protein or peptide is a L2 protein from a HPV16. In one example, the viral DNA-binding protein or peptide is a protein VII from an Ad5.

In one example, the viral DNA-binding protein or peptide is a nucleoprotein, wherein the DNA-binding protein or peptide encapsulates the DNA, stabilizes the DNA and inhibits induction of signalling by one or more endosomal Toll-like receptors (e.g., TLR-3, TLR-7, TLR-8 and/or TLR-9).

In one example, the viral DNA-binding protein or peptide is a nucleocapsid, wherein the DNA-binding protein or peptide encapsulates the DNA, stabilizes the DNA and inhibits induction of signalling by one or more endosomal Toll-like receptors (e.g., TLR-3, TLR-7, TLR-8 and/or TLR-9).

In one example, the viral DNA-binding protein or peptide is a matrix protein, wherein the DNA-binding protein or peptide binds to the DNA, stabilizes the DNA, but does not inhibit induction of signalling by one or more endosomal Toll-like receptors (e.g., TLR-3, TLR-7, TLR-8 and/or TLR-9).

In one example, the DNA-binding protein or peptide is a non-viral DNA-binding protein or peptide. For example, the DNA-binding protein or peptide is a non-viral protein or peptide derived from cellular proteins. In one example, the DNA-binding protein or peptide is derived from cellular proteins associated with cell growth, cell signalling and/or anti-viral pathways.

In one example, the cellular protein is selected from the group consisting of a TAR DNA binding protein (TRBP), Y-box binding protein, Z-DNA binding protein and combinations thereof.

In one example, the cellular protein is a TAR DNA binding protein (TRBP). In one example, the TRBP is TRBP 43.

In one example, the cellular protein is a Y-box binding protein. In one example, the Y-box binding protein is a Y-box binding protein 1.

In one example, the cellular protein is a Z-DNA binding protein. In one example, the cellular protein is a Z-DNA binding protein 1.

In one example, the lipid nanoparticle additionally comprises a PEG-lipid, a structural lipid and/or a neutral lipid. For example, the lipid nanoparticle additionally comprises a PEG-lipid, a structural lipid and a neutral lipid. In another example, the lipid nanoparticle additionally comprises a PEG-lipid, a structural lipid or a neutral lipid. In one example, the lipid nanoparticle additionally comprises a PEG-lipid, a structural lipid, an ionisable lipid and/or a neutral lipid. For example, the lipid nanoparticle additionally comprises a PEG-lipid, a structural lipid, an ionisable lipid and a neutral lipid. In another example, the lipid nanoparticle additionally comprises a PEG-lipid, a structural lipid, an ionisable lipid or a neutral lipid.

In one example, the lipid nanoparticle additionally comprises a PEG-lipid. For example, the PEG-lipid is selected from the group consisting of PEG-c-DMG, PEG-DMG, PEG-DLPE, PEG-DMPE, PEG-DPPC, a PEG-DSPE lipid and combinations thereof.

In one example, the lipid nanoparticle additionally comprises a structural lipid. For example, the structural lipid is selected from the group consisting of cholesterol, campesterol and combinations thereof.

In one example, the lipid nanoparticle additionally comprises a neutral lipid. For example, the neutral lipid is selected from the group consisting of DSPC, DOPE, DLPC, DMPC, DOPC, DPPC and combinations thereof.

In one example, the lipid nanoparticle additionally comprises an ionisable lipid. For example, the ionisable lipid is selected from the group consisting of: 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25),1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 1,2-dioleoyl-3-trimethylammonium propane (DOTAP), 1,2-distearyloxy-N,N-dimethyl-3-aminopropane (DSDMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-y loxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-die n-1-yloxy]propan-1-amine (Octyl-CLinDMA (2R)), (2S)-2-({8-[(3β)-cholest-5-en-3-yloxy]octyl}oxy)-N,N-dimethyl-3-[(9Z,12Z)-octadeca-9,12-die n-1-yloxy]propan-1-amine (Octyl-CLinDMA (2S)), 1,2-dilinolenyloxy-N,N-dimethyl-3-aminopropane (DLenDMA), 2,5-bis((9z,12z)-octadeca-9,12,dien-1-yloxyl)benzyl-4-(dimethylamino)butnoate (LKY750), 8-[(2-hydroxyethyl)[6-oxo-6-(undecyloxy)hexyl]amino]-octanoic acid, 1-octylnonyl ester (also referred to as heptadecan-9-yl 8-[2-hydroxyethyl-(6-oxo-6-undecoxyhexyl)amino]octanoate) (SM-102), 2-hexyl-decanoic acid, 1,1′-[[(4-hydroxybutyl)imino]di-6,1-hexanediyl]ester (also referred to as ((4-hydroxybutyl)azanediyl)bis(hexane-6,1-diyl)bis(2-hexyldecanoate)) (ALC-0315), 4-(dimethylamino)-butanoic acid, (10Z,13Z)-1-(9Z,12Z)-9,12-octadecadien-1-yl-10,13-nonadecadien-1-yl ester (DLin-MC3-DMA or MC3), ((4-hydroxybutyl)azanediyl)bis(hexane-6,1-diyl)bis(2-hexyldecanoate)), and 8-[(2-hydroxyethyl)[6-oxo-6-(undecyloxy)hexyl]amino]-octanoic acid, 1-octylnonyl ester and combinations thereof.

In one example, the lipid nanoparticle does not comprise an ionisable lipid.

In one example, the lipid nanoparticle does not comprise a cationic lipid.

In one example, the lipid nanoparticle comprises about 25 mol % to about 60 mol % compound of an ionisable lipid, about 2 mol % to about 25 mol % of a neutral lipid, about 18.5 mol % to about 60 mol % of a structural lipid, and about 0.2 mol % to about 10 mol % of a PEG-lipid, provided that the total mol % does not exceed 100%.

In one example, the lipid nanoparticle comprises about 10 mol % to about 50 mol % of an ionisable lipid, about 10 mol % to about 50 mol % of a neutral lipid, about 10 mol % to about 30 mol % of a structural lipid, and about 0.5 mol % to about 2.5 mol % of a PEG-lipid, provided that the total mol % does not exceed 100%.

In one example, the lipid nanoparticle comprises about 30 mol % to about 50 mol % compound of an ionisable lipid, about 5 mol % to about 20 mol % of a neutral lipid, about 30 mol % to about 55 mol % of a structural lipid, and about 1 mol % to about 5 mol % of a PEG-lipid, provided that the total mol % does not exceed 100%.

In one example, the lipid nanoparticle comprises about 40 mol % of an ionisable lipid, about 10 mol % of a neutral lipid, about 48 mol % of a structural lipid, and about 2.0 mol % of a PEG-lipid.

In one example, the lipid nanoparticles have a mean particle size of between about 80 nm and 200 nm. For example, the lipid nanoparticles have a mean particle size of between about 100 nm and 200 nm. In one example, the lipid nanoparticles have a mean particle size of between about 100 nm and 190 nm, or about 100 nm and 180 nm, or about 110 nm and 180 nm, or about 110 nm and 150 nm, or about 110 nm and 140 nm, or about 110 nm and 130 nm. For example, the lipid nanoparticles have a mean particle size of about 125 nm. In one example, the lipid nanoparticles have a mean particle size of between about 150 and 200 nm. In one example, the lipid nanoparticles have a mean particle size of between about 160 and 200 nm. For example, the lipid nanoparticles has a mean particle size of about 160 nm, or about 165 nm, or about 170 nm, or about 175 nm, or about 180 nm, or about 185 nm, or about 190 nm, or about 200 nm. In one example, the mean particle size is determined by measuring the Z-average diameter of the lipid nanoparticles.

In one example, the lipid nanoparticles have a nitrogen to phosphate ratio of between about 2 to about 10. For example, the lipid nanoparticles have a nitrogen to phosphate ratio of about 2, or about 2.5, or about 3, or about 3.5, or about 4, or about 4.5, or about 5, or about 5.5, or about 6, or about 6.5, or about 7, or about 7.5, or about 8, or about 8.5, or about 9, or about 9.5, or about 10. In one example, the lipid nanoparticles have a nitrogen to phosphate ratio of about 3. In another example, the lipid nanoparticles have a nitrogen to phosphate ratio of about 4.5. In a further example, the lipid nanoparticles have a nitrogen to phosphate ratio of about 6.

In one example, at least 50% of the RNA is encapsulated within the lipid nanoparticles. For example, at least 50%, or at least 55%, or at least 60%, or at least 65%, or at least 70%, or at least 75%, or at least 80%, or at least 85%, or at least 90%, or at least 95% of the RNA is encapsulated within the lipid nanoparticles. In one example, at least 80% of the RNA is encapsulated. In another example, at least 85% of the RNA is encapsulated. It will be apparent to the skilled person that encapsulation efficiency (or percent encapsulation) may be determined by measuring the escape or the activity of the pharmaceutical composition or mRNA of the disclosure using fluorescence (e.g., using RiboGreen) and/or electron micrograph.

In one example, the DNA is linear DNA. In one example, the DNA is non-linear DNA. For example, the non-linear DNA is a circular DNA, e.g., a plasmid or a covalently closed DNA. For example, as described in WO2010/086626 or WO2012/017210. In one example, the circular DNA is plasmid DNA. In another example, the DNA is covalently closed DNA.

The present disclosure also provides a composition comprising the lipid nanoparticle of the present disclosure. For example, the composition of the present disclosure, when administered, is capable of delivering the DNA to a cell, e.g., the nucleus of a cell in the subject. For example, the composition permits non-viral delivery of DNA to a cell, e.g., the nucleus of a cell in a subject.

The present disclosure also provides an immunogenic composition comprising the lipid nanoparticle of the present disclosure. For example, the composition of the present disclosure, when administered, is capable of inducing an immune response in the subject. For example, administration of the composition induces a humoral and/or a cell-mediated immune response. In one example, the composition induces a humoral immune response in the subject. For example, the humoral immune response is an antibody-mediated immune response. In another example, the composition induces a cell-mediated immune response. For example, the cell-mediated immune response includes activation of antigen-specific cytotoxic T cells.

The present disclosure also provides a pharmaceutical composition comprising an immunogenic composition of the present disclosure and a pharmaceutically acceptable carrier. Pharmaceutically acceptable carriers suitable for use in the present disclosure will be apparent to the skilled person and/or are described herein.

The present disclosure also provides the composition, immunogenic composition or the pharmaceutical composition of the disclosure for use in therapy. In one example, the therapy is gene therapy. For example, the immunogenic composition or the pharmaceutical composition of the disclosure is suitable for use as a vaccine.

In one example, the composition, immunogenic composition or the pharmaceutical composition of the disclosure is supplied in a vial. In another example, the immunogenic composition or the pharmaceutical composition of the disclosure is supplied in a syringe.

The present disclosure additionally provides a method for delivering DNA to the nucleus of a cell, the method comprising contacting the cell with the lipid nanoparticle described herein. For example, the disclosure provides a method for delivering DNA to a cell in a subject, the method comprising administering to the subject the lipid nanoparticle, composition or the pharmaceutical composition of the disclosure.

As will be apparent to the skilled person, the delivery of DNA to a subject permits a method of gene therapy. Thus, the disclosure provides a method of treating a subject comprising administering to the subject a lipid nanoparticle, composition or the pharmaceutical composition of the disclosure, wherein the DNA in the lipid nanoparticle, composition or the pharmaceutical composition of the disclosure encodes a therapeutic protein or an antigen.

### BRIEF DESCRIPTION OF THE DRAWINGS

FIG. 1 is a graphical representation showing SDS-PAGE of four nucleoproteins derived from influenza A, beak and feather disease virus (BFDV), Human Papillomavirus (HPV), and Hepatitis B (HBV).

FIG. 2 is a graphical representation showing HPLC-SEC of four nucleoproteins derived from influenza A, BFDV, HPV, and HBV.

FIG. 3 is a graphical representation showing EMSA of four nucleoproteins derived from influenza A, BFDV, HPV, and HBV with ssDNA.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

Any discussion of a protein or antibody herein will be understood to include any variants of the protein or antibody produced during manufacturing and/or storage. For example, during manufacturing or storage an antibody can be deamidated (e.g., at an asparagine or a glutamine residue) and/or have altered glycosylation and/or have a glutamine residue converted to pyroglutamate and/or have a N-terminal or C-terminal residue removed or “clipped” and/or have part or all of a signal sequence incompletely processed and, as a consequence, remain at the terminus of the antibody. It is understood that a composition comprising a particular amino acid sequence may be a heterogeneous mixture of the stated or encoded sequence and/or variants of that stated or encoded sequence.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### Selected Definitions

As used herein, the term “lipid nanoparticle” or “LNP” shall be understood to refer to lipid-based particles having at least one dimension on the order of nanometers (e.g., 1-1,000 nm) and which comprises a compound of any formulae described herein. In some examples, LNPs are formulated in a composition for delivery of a polynucleotide to a desired target such as a cell, tissue, organ, tumor, and the like. For example, the lipid nanoparticle or LNP may be selected from, but not limited to, liposomes or vesicles, where an aqueous volume is encapsulated by amphipathic lipid bilayers (e.g., single; unilamellar or multiple; multilamellar), micelle-like lipid nanoparticles having a non-aqueous core and solid lipid nanoparticles, wherein solid lipid nanoparticles lack lipid bilayers.

The term “lipidated” or “lipidation” as used herein refers to the process of covalently modifying a protein (i.e., a DNA-binding protein or peptide) with one or more lipids.

As used herein, the term “DNA-binding protein or peptide” or “DBP’ shall be understood to refer to proteins and peptides that bind to double or single stranded DNA and participate in forming deoxyribonucleoprotein complexes.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulfide bond. Examples of non-covalent bonds include hydrogen bonds, ionic bonds, Van der Waals forces, and hydrophobic interactions.

The term “peptide” as used herein is intended to include compounds composed of amino acid residues linked by amide bonds. A peptide may be natural or unnatural, ribosome encoded or synthetically derived. Typically, a peptide will consist of between 2 and 200 amino acids. For example, the peptide may have a length in the range of 10 to 20 amino acids or 10 to 30 amino acids or 10 to 40 amino acids or 10 to 50 amino acids or 10 to 60 amino acids or 10 to 70 amino acids or 10 to 80 amino acids or 10 to 90 amino acids or 10 to 100 amino acids, including any length within said range(s).

As used herein, the term “recombinant” shall be understood to mean the product of artificial genetic recombination.

As used herein, the term “subject” shall be taken to mean any animal including humans, for example a mammal. Exemplary subjects include but are not limited to humans and non-human primates. For example, the subject is a human.

### Lipid Nanoparticles

The present disclosure provides a lipid nanoparticle for delivery of DNA, wherein the lipid nanoparticle comprises a lipidated DNA-binding protein or peptide bound to the DNA.

### DNA-Binding Proteins or Peptides

The present disclosure provides a lipid nanoparticle comprising a DNA-binding protein or peptide. For example, the present disclosure provides a lipid nanoparticle comprising a lipidated DNA-binding protein or peptide.

DNA-binding proteins regulate numerous aspects of co- and post-transcription gene expression including, for example, DNA replication, DNA recombination, DNA modification, DNA repair, DNA organization/compaction, DNA storage and DNA stabilization. DNA-binding proteins or peptide bind to double or single-stranded DNA and participate in the formation of deoxyribonucleoprotein complexes. The skilled person will understand that DNA-binding proteins or peptides can be viral or non-viral proteins or peptides.

### Non-Viral DNA-Binding Proteins or Peptides

In one example, the DNA-binding protein is a non-viral protein or peptide derived from cellular proteins. For example, DNA-binding proteins or peptides are derived from cellular proteins associated with cell growth, cell signalling and/or anti-viral pathways.

Non-viral DNA-binding proteins or peptides contain numerous structural motifs or DNA-binding domains that facilitate DNA binding including, for example, a helix-turn-helix (HTH) motif, a basic helix-loop-helix (bHLH) domain, a zinc finger (ZnF) domain, a leucine zipper (bZIP) domain, a winged helix (WH), a winged helix-turn-helix (wHTH) domain, a β-sheet motif, a β-hairpin/ribbon motif, a high mobility group (HMG) domain, a Wor3 domain, a GB-fold domain, a immunoglobulin domain, a B3 domain, a TAL effector, homeodomain motif, histone fold, AT-hook domain, TATA binding protein (TBP) domain, histone-like protein (HU) motif, POU domain, Zn-containing motifs, and receptor DNA-binding domain (DBD).

In one example, the DNA-binding protein or peptide comprises a HTH motif. For example, the DNA-binding protein or peptide comprising a HTH motif is selected from the group consisting of 1LMB, 1LLI, 1PER, 1RPE, 20R1, 3CRO, 6CRO, 6CRO, 30RC, 1WT, 1BDH, 1BDI, 1PNR, 2PUA, 2PUB, 2PUC, 2PUD, 2PUE, 2PUF, 2PUG, QVPW, 1QPZ, 1ZAY, 1FOK, 1GDT, 1HCR, 1IGN, 1PDN, 1TC3, 1TRR, 1DDN, 1D3U, 1VOL and 1C9B.

In one example, the DNA-binding protein or peptide comprises a bHLH motif. For example, the DNA-binding protein or peptide comprising a bHLH motif is selected from the group consisting of 1AM9, 1HLO, 1AN4, 1AN2, 1MDY and 1AOA.

In one example, the DNA-binding protein or peptide comprises a ZnF motif. For example, the DNA-binding protein or peptide comprising a ZnF motif is selected from the group consisting of 1AAY, 1ZAA, 2DRP, 1UBD, 1MEY, 1A1G, 1A1H, 1A1I, 1A1J, 1A1K, 1A1L, 2GLI, 2NLL, 1HCQ, 1GLU, 1LAT, 1BY4, 1CIT, 1A6Y, 1TSR, 1TUP, 1ZME and 1D66.

In one example, the DNA-binding protein or peptide comprises a bZIP motif. For example, the DNA-binding protein or peptide comprising a bZIP motif is selected from the group consisting of 2DGC, 1DGC, 1YSA and 1A02.

In one example, the DNA-binding protein or peptide comprises a WH motif. For example, the DNA-binding protein or peptide comprising a WH motif is selected from the group consisting of 2IRF, 1IF1, 2CGP, 1BER, 1CGP, 1RUN, 1RUO, 3HTS, 1CF7, 1BC8, 1BC7, 1PUE and 1AWC.

In one example, the DNA-binding protein or peptide comprises a homeodomain motif. For example, the DNA-binding protein or peptide comprising a homeodomain motif is selected from the group consisting of 1FJ1, 1HDD, 1APL, lYRN, 1AU7, 1OCT, 2HDD, 3HDD, 9ANT, 6PAX, 1AKH, 1B72, 1B8I and 1MNM.

In one example, the DNA-binding protein or peptide comprises a j-sheet motif. For example, the DNA-binding protein or peptide comprising a j-sheet motif is selected from the group consisting of 1TYB, 1YTF, 1AIS, 1CDW, 1TGH, 1VOL, 1D3Y, 1C9B,

In one example, the DNA-binding protein or peptide comprises a β-hairpin/ribbon motif. For example, the DNA-binding protein or peptide comprising a β-hairpin/ribbon motif is selected from the group consisting of 1CMA, 1ECR, 1IHF, 1XBR, 1AZP, 1BNZ, 1BF4, 1BDT, 1BDV and 1PAR.

### Viral DNA-Binding Proteins

In one example, the DNA-binding protein or peptide is a viral DNA-binding protein or peptide. For example, the DNA-binding protein is a nucleoprotein, a matrix protein, a nucleocapsid protein and/or a non-structural from a DNA virus.

It will be apparent to the skilled person that viruses are classified according to the Baltimore classification system, as shown in Table 1, which is largely based on the transcription of the viral genome.

TABLE 1
Baltimore classification of viruses

| Class | Genome |
|---|---|
| I | double-stranded DNA viruses |
| II | single-stranded DNA viruses |
| III | double-stranded RNA viruses |
| IV | positive sense single-stranded RNA viruses |
| V | negative sense single-stranded RNA viruses |
| VI | single-stranded RNA viruses with a DNA intermediate in their |
| | life cycle |
| VII | double-stranded DNA viruses with a RNA intermediate in their |
| | life cycle |

In one example, the DNA-binding protein or peptide is from a DNA virus. For example, the DNA-binding protein or peptide is from a class I, a class II, and/or a class VII virus.

In one example, the DNA virus is a class I virus (i.e., a double-stranded DNA virus). Class I viruses include, for example, all viruses of the realm Duplodnaviria, Adnaviria, and Varidnaviria, all viruses of class Papovaviricetes (of realm Monodnaviria), and Naldaviricetes, all viruses of the family Ampullaviridae, Bicaudaviridae, Clavaviridae, Fuselloviridae, Globuloviridae, Guttavi ridae, Halspiviridae, Ovaliviridae, Plasmaviridae, Polydnaviridae, Portogloboviridae, and Thaspiviridae, and all viruses of the genera Dinodnavirus and Rhizidiovirus. Exemplary class I viruses include, but are not limited to, adenoviruses (e.g., Ad5), herpesviruses, human papillomavirus (e.g., HPV16) and poxviruses.

In one example, the DNA virus is a class II virus (i.e., a single-stranded DNA virus). Class II viruses include, for example, viruses of the realm Monodnaviria (except of the class Papovaviricetes) and of families Anelloviridae, Circoviridae, Spiraviridae and Finnlakeviridae. Exemplary class II viruses include, but are not limited to, beak and feather disease virus, adeno-associated viruses, geminiviruses, bacteriophages and parvoviriuses.

In one example, the DNA virus is a class VII virus (i.e., single-stranded DNA viruses with an RNA intermediate in their life cycle). Class VII viruses include, for example, viruses of the family Caulimoviridae (of phylum Aterviricota). Exemplary class VII viruses include, but are not limited to, heparnaviruses (e.g., Hepatitis A virus (HAV)) and hepadnaviruses (e.g., Hepatitis B virus, Hepatitis C virus).

### Linkers

In one example, the DNA-binding protein or peptide comprises a first DNA-binding protein or peptide and a second DNA-binding protein or peptide linked via a linker. For example, the linker is a linker peptide.

In one example, the linker is a flexible linker.

A “flexible” linker is an amino acid sequence which does not have a fixed structure (secondary or tertiary structure) in solution. Such a flexible linker is therefore free to adopt a variety of conformations. Flexible linkers suitable for use in the present disclosure are known in the art. An example of a flexible linker for use in the present disclosure is the linker sequence SGGGGS/GGGGS/GGGGS or (Gly₄Ser)₃. Another example of a flexible linker is an alanine linker (e.g., Alaₙ).

The linker may comprise any amino acid sequence that does not substantially hinder interaction of the DNA-binding protein or peptide with the DNA. Preferred amino acid residues for flexible linker sequences include, but are not limited to, glycine, alanine, serine, threonine proline, lysine, arginine, glutamine and glutamic acid.

The linker sequences between the DNA-binding protein or peptide preferably comprise five or more amino acid residues. The flexible linker sequences according to the present disclosure consist of 5 or more residues, preferably, 5, 6, 7, 8, 9, 10, 11, 12, 13, 14, 15, 16, 17, 18, 19 or 20 or more residues. In an example, the flexible linker sequences consist of 5, 7, 10 or 16 residues.

In one example, the linker is a rigid linker. A “rigid linker” (including a “semi-rigid linker”) refers to a linker having limited flexibility. For example, the relatively rigid linker comprises the sequence (EAAAK)ₙ, where n is between 1 and 3. The value of n can be between 1 and about 10 or between about 1 and 100. For example, n is at least 1, or at least 2, or at least 3, or at least 4, or at least 5, or at least 6, or at least 7, or at least 8, or at least 9, or at least 10. In one example, n is less than 100. For example, n is less than 90, or less than about 80, or less than about 70, or less than about 60, or less than about 50, or less than about 40, or less than about 30, or less than about 20, or less than about 10. A rigid linker need not completely lack flexibility.

### Lipidation of the DNA-Binding Protein

The present disclosure provides a lipid nanoparticle comprising a lipidated DNA-binding protein or peptide.

It will be apparent to the skilled person that protein or peptide lipidation is the covalent attachment of a lipid moiety to the protein or peptide (i.e., DNA-binding protein or peptide).

### Lipids

Lipid moieties suitable for use in the present disclosure will be apparent to the skilled person and include, for example, a fatty acid, an isoprenoid and combinations thereof. In one example, the lipid moiety is selected from the group consisting of an isoprenoid, a triglyceride, a phospholipid, a cholesteryl ester and combinations thereof.

### Isoprenoids

Isoprenoids, also known as terpenoids or prenol lipids, are branched lipids and are a class of organic compounds composed of two or more units of hydrocarbons, with each unit consisting of five carbon atoms arranged in a specific pattern. These five-carbon units are termed isoprene and are synthesized from a common intermediate known as mevalonic acid, which is itself synthesized from acetyl-CoA. Isoprenoids can have one or more functional chemical groups attached to their carbon backbone, such as hydroxyls and carbonyls, which make up the diversity of isoprenoids. Isoprenoids can be classified as monoterpenes (C₁₀H₁₆) sesquiterpenes (C₁₅H₂₄), diterpenes (C₂₀H₃₂), triterpenes (C₃₀H₄₈), tetraterpenes (C₄₀H₆₄) or other polyterpenes (C₅H₈)ₙ.

Isoprenoids suitable for use in the present disclosure will be apparent to the skilled person and/or are described herein.

In one example, the isoprenoid is a monoterpene. Exemplary monoterpenes include citronellol, citronellal, citral, geraniol, methol, pseudoionone and beta-ionone.

In one example, the isoprenoid is a sesquiterpenes. Exemplary sesquiterpenes include cadalene, eudalene, cadinene and beta-selinene.

In one example, the isoprenoid is a diterpene. Exemplary diterpenes include phytol and abietic acid.

In one example, the isoprenoid is a triterpene. Exemplary triterpenes include squalene and beta-amyrin.

In one example, the isoprenoid is a tetraterpene. Exemplary tetraterpenes include carotenoids (e.g., beta-carotene) and lycopene.

### Fatty Acids

Fatty acids are lipids that contain long-chain hydrocarbons terminated with a carboxylic acid functional group. Fatty acids may be saturated or unsaturated. In one example, the fatty acid comprises a carbon chain having from 6 to 22 carbons. Exemplary fatty acids include palmitic acid, myristic acid, oleic acid, alpha-linolenic acid and stearic acid.

Fatty acids rarely occur in the free form in nature and commonly exist as three main classes of esters: triglycerides, phospholipids and cholesteryl esters.

In one example, the fatty acid is a triglyceride. Triglycerides are tri-esters consisting of a glycerol bound to three fatty acid molecules via an ester bond. The three fatty acids may be the same or different. An exemplary triglyceride is tristearin.

In one example, the fatty acid is a phospholipid. Phospholipids are complex lipids that comprise a hydrophilic polar head group comprising one or more phosphate groups, and a hydrophobic tail comprising two fatty acyl chains. The polar head group is joined to the hydrophobic moiety by a phosphodiester linkage via a glycerol (i.e., phosphoglycerides) or sphingosine molecule (i.e., phosphosphingo lipids). Phospholipids may be saturated or unsaturated. Exemplary phosphoglycerides include phosphatidic acid (phosphatidate), phosphatidylethanolamine (cephaline), phosphatidylcholine (lecithin), phosphatidylserine, phosphoinositides (e.g., phosphatidylinositol (PI), phosphatidylinositol phosphate (PIP), phosphatidylinositol bisphosphate (PIP2) and phosphatidylinositol trisphosphate (PIP3)), phosphatidylglycerol and cardiolipin. Exemplary phosphosphingo lipids include ceramide phosphorylcholine (sphingomyelin), ceramide phosphorylethanolamine (sphingomyelin), ceramind phosphoryllipid, galactocerebroside, glucocerebroside and lactosylceramide.

In one example, the fatty acid is a cholesteryl ester. Cholesteryl esters are the esterification of cholesterol with long-chain fatty acids. Exemplary cholesteryl esters include cholesteryl oleate, cholesteryl benzoate and cholesteryl linoleate.

### Lipidation

Exemplary lipidation includes, palmitoylation, myristoylation, fatty-acylation, esterification, prenylation, or combinations thereof.

### Palmitoylation

In one example, the lipid moiety is attached to the DNA-binding protein or peptide by palmitoylation.

In one example, the palmitoylation is cysteine palmitoylation (also known as S-palmitoylation). The skilled person will understand that cysteine palmitoylation is the addition of a 16-carbon palmitoyl group on protein cysteine residues. In one example, the palmitoyl group is added via a thioester bond. In another example, the palmitoyl group is added via an amide bond.

### Myristoylation

In one example, the lipid moiety is attached to the DNA-binding protein or peptide by myristoylation.

In one example, the myristoylation is N-glycine myristoylation. The skilled person will recognise that N-glycine myristoylation refers to the co- or post-translational attachment of a saturated 14-carbon fatty acyl group, myristoyl, to the N-terminal glycine of proteins via an amide bond.

In one example, the myristoylation is lysine myristoylation.

### Fatty-Acylation

In one example, the lipid moiety is attached to the DNA-binding protein or peptide by fatty-acylation.

The skilled person will recognise that fatty-acylation involves the covalent attachment of an acyl group to a protein.

In one example, the fatty-acylation is lysine N-acylation. The skilled person will understand that lysine N-acylation refers to the transfer of the acetyl moiety from acetyl-CoA to the epsilon (ε)-amino group of a lysine residue on a protein.

### Esterification

In one example, the lipid moiety is attached to the DNA-binding protein or peptide by esterification.

In one example, the esterification is C-terminal sterol esterification, for example C-terminal cholesterol esterification. The skilled person will understand that C-terminal cholesterol esterification is the replacement of at least one hydroxyl (—OH) group with an alkoxy (—O-alkyl) group.

### Prenylation

In one example, the lipid moiety is attached to the DNA-binding protein or peptide by prenylation.

In one example, the prenylation is cysteine prenylation. The skilled person will understand that cysteine prenylation is the addition of multiple isoprene units to cysteine residues near the C-terminal end of the protein.

In one example, the prenylation is farnesylation (i.e., the addition of three isoprene units), or the prenylation is geranylgeranylation (i.e., the addition of four isoprene units).

In one example, the linkage between farnesyl or geranylgeranyl groups and cysteine residues is a thioether bond. In another example, the linkage is an ester bond. In a further example, the linkage is a thioester bond.

### Methods of Lipidation

Lipid modifications typically occur on the nucleophilic side chains of proteins or peptide (e.g., cysteine, serine and lysine), at the N-terminal end and/or at the C-terminal end of proteins or peptides.

Various methods of lipidation will be apparent to the skilled person and/or are described herein. Suitable methods can include chemical or enzymatic lipidation.

### Chemical Lipidation

In one example, the lipid moiety is attached to the DNA-binding protein or peptide using chemical ligation. The lipid moiety can comprise an amine, carboxylic acid, hydrazide, or maleimide group and the lipid moiety may be chemically coupled to the DNA-binding protein or peptide via the primary amine group of a lysine or the thiol group of a cysteine. In one example, the lipid moiety comprises a maleimide group and the lipid moiety is attached to the DNA-binding protein or peptide via the formation of a thioether bond with a sulphydryl group in the DNA binding protein or peptide. In one example, the lipid moiety comprises a carboxylic acid and the carboxylic acid is activated by 1-ethyl-3-(3-dimethylaminopropyl) carbodiimide (EDC) and N-hydroxysulfosuccinimide (Sulfo-NHS). The activated acyl amino ester or sulfo-NHS ester subsequently reacts with a primary amine of a lysine residue in the DNA-binding protein or peptide forming an amide bond.

In one example, the lipid moiety comprises a maleimide group. For example, the lipid moiety is a phospholipid capped with a maleimide group. In one example, the lipid moiety is a 1,2-Distearoyl-sn-Glycero-3-Phosphoethanolamine-maleimide (DSPE-maleimide; DSPE-Mal).

In one example, the lipid moiety is attached to the DNA-binding protein or peptide using various “click chemistry” strategies such as those disclosed in Kolb et al. (2001), WO 2003/101972 and Malkoch et al. (2005).

In one example, the lipid moiety is attached to the DNA-binding protein or peptide using expressed protein ligation. Expressed protein ligation comprises chemoselective ligation between a protein or peptide with a C-terminal thioester and a protein or peptide with an N-terminal cysteine in aqueous solution at physiological pH. In one example, the C-terminal thioester is inserted into the DNA-binding protein or peptide by genetic manipulation and the lipid moiety is fused to a peptide having an N-terminal cysteine residue.

Other methods of chemical lipidation known to the skilled person may be used, such as those disclosed in Takahara & Kamiya (2020).

### Enzymatic Lipidation

In one example, the lipid moiety is attached to the DNA-binding protein or peptide using enzymatic lipidation. Enzymatic lipidation may be performed in vivo or in vitro. In some examples, the DNA-binding protein or peptide is genetically manipulated using techniques known to the skilled person to comprise a consensus sequence recognized by the lipidating enzyme.

In one example, the lipid moiety is attached to the DNA-binding protein or peptide using Sortase-A mediated lipidation. Sortase A (e.g., SrtA from Staphylococcus aureus) covalently attaches secreted proteins to a bacterial cell wall peptidoglycan in the presence of Ca²⁺ via a transpeptidation reaction. In this example, the DNA-binding protein or peptide is genetically manipulated to comprise an LPXTG motif (e.g., LPETG) at the C-terminus and the lipid moiety comprises a nucleophile and an oligo-glycine motif (e.g., triglycine, tetraglycine or pentaglycine). Upon addition of the sortase, the DNA-binding protein or peptide is covalently linked to the lipid through a peptide bond.

In one example, the lipid moiety is attached to the DNA-binding protein or peptide using transglutaminase mediated lipidation. Transglutaminase (e.g., Microbial transglutaminase: MTG) catalyzes a reaction between a glutamine residue and a lysine residue in a peptide or protein in the absence Ca²⁺ forming an irreversible cross-link. In one example, the DNA-binding protein or peptide is genetically manipulated to comprise the MTG lysine recognition sequence (e.g., MRHKGS), for example at the N- or C-terminus, and the lipid moiety comprises the MTG glutamine recognition sequence (e.g., LLQG). In one example, the DNA-binding protein or peptide is genetically manipulated to comprise the MTG glutamine recognition sequence (e.g., LLQG or LQ), for example at the N- or C-terminus, and the lipid moiety comprises MTG lysine recognition sequence (e.g., MRHKGS).

Other methods of enzymatic lipidation known to the skilled person may be used, such as those disclosed in Takahara & Kamiya (2020).

### Additional Lipids

In one example, the lipid nanoparticle additionally comprises a PEG-lipid, a sterol structural lipid and/or a neutral lipid. In one example, the lipid nanoparticle additionally comprises a PEG-lipid, a sterol structural lipid, an ionisable lipid and/or a neutral lipid. In one example, the lipid nanoparticle does not comprise a cationic lipid.

### PEG-Lipids

In one example, the present disclosure provides a lipid nanoparticle comprising a PEGylated lipid.

It will be apparent to the skilled person that reference to a PEGylated lipid is a lipid that has been modified with polyethylene glycol. Exemplary PEGylated lipids include, but are not limited to, PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid includes PEG-c-DMG, PEG-DMG, PEG-DLPE, PEG-DMPE, PEG-DPPC, a PEG-DSPE lipid and combinations thereof.

### Neutral Lipids

In one example, the present disclosure provides a lipid nanoparticle comprising a neutral lipid.

Suitable neutral or zwitterionic lipids for use in the present disclosure will be apparent to the skilled person and include, for example, 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine (DSPE), 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. The lipids can be saturated or unsaturated.

### Structural Lipids

In one example, the present disclosure provides a lipid nanoparticle comprising a structural lipid.

Exemplary structural lipids include, but are not limited to, cholesterol fecosterol, sitosterol, campesterol, stigmasterol, brassicasterol, ergosterol, tomatidine, tomatine, ursolic acid and alpha-tocopherol.

In one example, the structural lipid is a sterol. For example, the structural lipid is cholesterol. In another example, the structural lipid is campesterol.

### Ionisable Lipids

In one example, the present disclosure provides a lipid nanoparticle comprising an ionisable lipid.

Suitable ionisable lipids for use in the present disclosure will be apparent to the skilled person and include, for example, 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25),1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 1,2-dioleoyl-3-trimethylammonium propane (DOTAP), 1,2-distearyloxy-N,N-dimethyl-3-aminopropane (DSDMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-y loxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-die n-1-yloxy]propan-1-amine (Octyl-CLinDMA (2R)), (2S)-2-({8-[(3β)-cholest-5-en-3-yloxy]octyl}oxy)-N,N-dimethyl-3-[(9Z,12Z)-octadeca-9,12-die n-1-yloxy]propan-1-amine (Octyl-CLinDMA (2S)), 1,2-dilinolenyloxy-N,N-dimethyl-3-aminopropane (DLenDMA), 2,5-bis((9z,12z)-octadeca-9,12,dien-1-yloxyl)benzyl-4-(dimethylamino)butnoate (LKY750), 8-[(2-hydroxyethyl)[6-oxo-6-(undecyloxy)hexyl]amino]-octanoic acid, 1-octylnonyl ester (also referred to as heptadecan-9-yl 8-[2-hydroxyethyl-(6-oxo-6-undecoxyhexyl)amino]octanoate) (SM-102), 2-hexyl-decanoic acid, 1,1′-[[(4-hydroxybutyl)imino]di-6,1-hexanediyl]ester (also referred to as ((4-hydroxybutyl)azanediyl)bis(hexane-6,1-diyl)bis(2-hexyldecanoate)) (ALC-0315), 4-(dimethylamino)-butanoic acid, (10Z,13Z)-1-(9Z,12Z)-9,12-octadecadien-1-yl-10,13-nonadecadien-1-yl ester (DLin-MC3-DMA or MC3), ((4-hydroxybutyl)azanediyl)bis(hexane-6,1-diyl)bis(2-hexyldecanoate)), and 8-[(2-hydroxyethyl)[6-oxo-6-(undecyloxy)hexyl]amino]-octanoic acid, 1-octylnonyl ester and combinations thereof.

### Pharmaceutically Acceptable Carrier

Suitably, in compositions or methods for administration of the lipid nanoparticle of the disclosure to a subject, the lipid nanoparticle is combined with a pharmaceutically acceptable carrier as is understood in the art. Accordingly, one example of the present disclosure provides a composition (e.g., a pharmaceutical composition) comprising the lipid nanoparticle of the disclosure combined with a pharmaceutically acceptable carrier.

In general terms, by “carrier” is meant a solid or liquid filler, binder, diluent, encapsulating substance, emulsifier, wetting agent, solvent, suspending agent, coating or lubricant that may be safely administered to any subject, e.g., a human. Depending upon the particular route of administration, a variety of acceptable carriers, known in the art may be used, as for example described in Remington's Pharmaceutical Sciences (Mack Publishing Co. N.J. USA, 1991).

A lipid nanoparticle of the present disclosure is useful for parenteral, topical, oral, or local administration, intramuscular administration, aerosol administration, or transdermal administration, for prophylactic or for therapeutic treatment. In one example, the lipid nanoparticle is administered parenterally, such as intramuscularly, subcutaneously or intravenously. For example, the lipid nanoparticle is administered intramuscularly.

Formulation of lipid nanoparticle to be administered will vary according to the route of administration and formulation (e.g., solution, emulsion, capsule) selected. An appropriate pharmaceutical composition comprising a lipid nanoparticle to be administered can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The lipid nanoparticle can be stored in the liquid stage or can be lyophilized for storage and reconstituted in a suitable carrier prior to use according to art-known lyophilization and reconstitution techniques.

The optimum concentration of the active ingredient(s) (i.e., the DNA) in the chosen medium can be determined empirically, according to procedures known to the skilled artisan, and will depend on the ultimate pharmaceutical formulation desired.

Upon formulation, compositions of the present disclosure will be administered in a manner compatible with the dosage formulation and in such amount as is therapeutically/prophylactically effective. The dosage ranges for the administration of the lipid nanoparticle of the disclosure are those large enough to produce the desired effect. For example, the composition comprises an effective amount of the encapsulated DNA. In one example, the composition comprises a therapeutically effective amount of the DNA. In another example, the composition comprises a prophylactically effective amount of the DNA.

The dosage should not be so large as to cause adverse side effects. Generally, the dosage will vary with the age, condition, sex and extent of the disease in the patient and can be determined by one of skill in the art. The dosage can be adjusted by the individual physician in the event of any complication.

### DNA

The present disclosure provides a lipid nanoparticle for delivery of DNA, wherein a DNA-binding protein or peptide is bound to the DNA. For example, the present disclosure provides a lipid nanoparticle for delivery of DNA, wherein a lipidated DNA-binding protein or peptide is bound to the DNA.

The DNA of the present disclosure may be a naturally or non-naturally occurring DNA, or may include one or more modified nucleobases, nucleosides, nucleotides, a promoter, an enhancer (e.g., cytomegalovirus), a poly(A) sequence or polyadenylation signal.

### Methods of Preparation

Suitable methods for the production of a lipid nanoparticle of the present disclosure will be apparent to the skilled person and/or described herein. For example, a lipid nanoparticle of the present disclosure may be made using approaches which are well-known in the art of formulation. For example, suitable LNPs can be formed using mixing processes such as microfluidics, including herringbone micromixing, and T-junction mixing of two fluid streams, one of which contains the DNA, typically in an aqueous solution, and the other of which has the various required lipid components, typically in ethanol.

The LNPs may then be prepared by combining a phospholipid (such as DOPE or DSPC, which may be purchased from commercial sources including Avanti Polar Lipids, Alabaster, AL), a PEGylated lipid (such as 1,2-dimyristoyl-sn-glycerol methoxypoly ethylene glycol, also known as PEG-DMG, which may be purchased from commercial sources including Avanti Polar Lipids, Alabaster, AL), and a structural lipid/sterol (such as cholesterol, which may be purchased from commercial sources including Sigma-Aldrich), at concentrations of, for example, about 50 mM in ethanol. Solutions should be refrigerated during storage at, for example, −20° C. The various lipids may be combined to yield the desired molar ratios and diluted with water and ethanol to a final desired lipid concentration of, for example, between about 5.5 mM and about 25 mM.

An LNP composition comprising a DNA, including, but not limited to, as a ssDNA or dsDNA, may prepared by combining the above lipid solution with a solution including the DNA at, for example, a lipid component to DNA wt:wt ratio from about 5:1 to about 50:1. The lipid solution may be rapidly injected using a NanoAssemblr microfluidic system at flow rates between about 3 ml/min and about 18 ml/min into the DNA solution to produce a suspension with a water to ethanol ratio between about 1:1 and about 4:1.

For LNP compositions including ssDNA or dsDNA, solutions of the DNA at concentrations of 1.0 mg/ml in deionized water may be diluted in 50 mM sodium citrate buffer at a pH between 3 and 6 to form a stock solution.

LNP compositions may be further processed, as is known in the art, by 10-fold dilution into 50 mM citrate buffer at pH 6 and subjected to tangential flow filtration (TFF) using a 300 k molecular weight cut-off membrane (mPES) until concentrated to the original volume. Subsequently, in one example, the citrate buffer may be replaced with a buffer containing 20 mM Tris buffer at pH 7.5, 80 mM sodium chloride, and 3% sucrose using diafiltration with a 10-fold volume of the new buffer. The LNP solution may be concentrated to a volume of, for example, between 5-10 mL, filtered using a 0.2 micron PES syringe filter, aliquoted into vials, and frozen at 1° C./min using a Corning® CoolCell® LX Cell Freezing Container until the samples reach −80° C. Samples may be stored at −80° C. until needed.

The method described above induces nano-precipitation and particle formation. Alternative processes including, but not limited to, T-junction and direct injection, may be used to achieve the same nano-precipitation.

In some examples, the lipid component of the LNP formulation comprises about 2 mol % to about 25 mol % phospholipid (neutral lipid), about 18.5 mol % to about 60 mol % structural lipid (sterol), and about 0.2 mol % to about 10 mol % of PEGylated lipid, provided that the total mol % does not exceed 100%. In some examples, the lipid component of the LNP formulation comprises about 5 mol % to about 20 mol % phospholipid, about 30 mol % to about 55 mol % structural lipid, and about 1 mol % to about 5 mol % of PEGylated lipid. In a particular examples, the lipid component includes about 10 mol % phospholipid, about 48 mol % structural lipid, and about 2.0 mol % of PEG lipid. In some examples, the phospholipid may be DOPE or DSPC. In other examples, the PEG lipid may be PEG-DMG and/or the structural lipid may be cholesterol.

The efficiency of encapsulation of the DNA within the LNPs may be at least 50%, for example about 50%, 55%, 60%, 65%, 70%, 75%, 80%, 85%, 90%, 91%, 92%, 93%, 94%, 95%, 96%, 97%, 98%, or 99%. In some examples, the encapsulation efficiency may be at least 80%. In certain examples, the encapsulation efficiency may be at least 90%.

### Assaying Lipid Nanoparticles of the Disclosure

Lipid nanoparticles of the present disclosure are readily screened for physical and biological activity and/or stability using methods known in the art and/or as described below.

### Assessing DNA Degradation

In one example, the level of DNA degradation by DNases is assessed. For example, the DNA, alone or in combination with the DNA-binding protein or peptide, is treated with DNase.

In one example, the level of DNA is assessed in DNAse treated and untreated samples using real time PCR. In one example, the cycle threshold (CT) value in DNA samples without a DNA-binding protein or peptide are increased compared to DNA samples with a DNA-binding protein or peptide indicating DNA degradation.

### Assessing DNA Translation

In one example, DNA translation is assessed using a cell based reporter system. Suitable systems for use in the present disclosure will be apparent to the skilled person, and include for example the use of a cell-based fluorescent reporter system or histochemical expression of alkaline phosphatase.

In one example, the cell based reported system comprises delivery of a DNA reporter for expression of an enzyme that leads to conversion of a substrate that can be visualised by microscopy or quantitated by colorimetric analysis.

In one example, the DNA is assessed in the presence or absence of a DNA-binding protein or peptide.

In one example, the DNA is nanoluciferase DNA and the amount of DNA translation is measured by the amount of luciferase produced as assessed by measuring luminescence in relative light units (RLU). In one example, the assay is performed at 4° C., 24° C. and/or 37° C. In another example, the assay is performed after incubating the samples for 0 hours, 1 hour, 2 hours, 4 hours, 8 hours, 24 hours, 48 hours or 96 hours.

### Assessing TLR Induction

In one example, the level of TLR-3, TLR-7, TLR-8 and/or TLR-9 induction is assessed. For example, the level of TLR-3, TLR-7, TLR-8 and/or TLR-9 induction by the DNA, alone or in combination with the DNA-binding protein or peptide is assessed. In one example, TLR-3, TLR-7, TLR-8 and/or TLR-9 induction is assessed using a TLR induction NfKB reporter assay. In this assay, NfKB is operationally linked to a secretary alkaline phosphatase (SEAP). DNA is introduced into either cell type (TLR-3 conditionally transduced, TLR-7 conditionally transduced, TLR-8 conditionally transduced or TLR-9 conditionally transduced). Binding of the TLR receptor induces NfKB activation and in turn SEAP. In one example, the SEAP level is determined by a chemical reaction and calorific read out.

### Use of Lipid Nanoparticles of the Disclosure

Lipid nanoparticles of the present disclosure can be used to deliver DNA to a cell by any of a number of methods and strategies known in the art, e.g., transient transfection, stable transfection, and viral transduction.

In one example, lipid nanoparticles of the present disclosure are used to deliver DNA to genetically modify a cell.

In one example, lipid nanoparticles of the present disclosure are used in combination with a second lipid nanoparticle comprising a nucleic acid encoding a programmable nuclease and a nucleic acid encoding a guide RNA (gRNA) to genetically modify a cell. In another example, lipid nanoparticles of the present disclosure are used in combination with a second lipid nanoparticle comprising a nucleic acid encoding a programmable nuclease and a third lipid nanoparticle comprising a nucleic acid encoding a gRNA to genetically modify a cell.

In one example, the nucleic acid is RNA. For example, the RNA is mRNA.

In one example, genetically modifying a cell includes repairing DNA breaks by endogenous cellular processes, such as homology-directed repair (HDR) and non-homologous end joining (NHEJ).

In one example, lipid nanoparticles of the present disclosure are used to deliver DNA to genetically modify a cell using HDR.

HDR is essentially an error-free mechanism that repairs double-strand DNA breaks in the presence of a homologous DNA sequence. The most common form of HDR is homologous recombination. It utilizes a homologous sequence as a template for inserting or replacing a specific DNA sequence at the break point. The template for the homologous DNA sequence can be an endogenous sequence (e.g., a sister chromatid), or an exogenous or supplied sequence (e.g., plasmid or an oligonucleotide). As such, HDR may be utilized to introduce precise alterations such as replacement or insertion at desired regions.

In one example, the DNA-binding protein or peptide bound to the DNA is a template for the homologous DNA sequence. In one example, the template comprises homology arms. In some examples, the homology arms are 100 nucleotides long.

In contrast, NHEJ is an error-prone repair mechanism that directly joins the DNA ends resulting from a double-strand break with the possibility of losing, adding or mutating a few nucleotides at the cleavage site. The resulting small deletions or insertions (termed “indels”) or mutations may disrupt or enhance gene expression. Additionally, if there are two breaks on the same DNA, NHEJ can lead to the deletion or inversion of the intervening segment. Therefore, NHEJ may be utilized to introduce insertions, deletions or mutations at the cleavage site.

In one example, the gRNA targets the globin locus. For example, the gRNA targets the α-globin locus. In another example, the gRNA targets the β-globin locus.

Programmable nucleases that can be used in accordance with the present disclosure include, but are not limited to, RNA-guided engineered nuclease (RGEN) derived from the bacterial clustered regularly interspaced short palindromic repeat (CRISPR)-cas, (CRISPR-associated) system, zinc-finger nuclease (ZFN), transcription activator-like nuclease (TALEN), meganucleases and argonautes.

In one example, the cells are ex vivo, e.g., as a cultured cell population. In another example, the cells are in vivo, e.g., in a mouse or human. In one example, the genetically modified cells are mammalian cells. For example, the mammalian cells are human cells.

In one example, the cells are haematopoietic stem cells. In one example, the cells are haematopoietic progenitor cells. In one example, the cells are haematopoietic stem and progenitor cells. For example, the cells are CD34+ haematopoietic stem and/or progenitor cells.

The invention is further disclosed in the following numbered paragraphs:
1. A lipid nanoparticle for delivery of DNA, the lipid nanoparticle comprising therein a DNA-binding protein or peptide bound to the DNA.
2. The lipid nanoparticle of paragraph 1, wherein the DNA-binding protein or peptide is a lipidated DNA-binding protein or peptide.
3. A lipid nanoparticle for delivery of DNA, the lipid nanoparticle comprising therein a lipidated DNA-binding protein or peptide bound to the DNA.
4. The lipid nanoparticle of paragraphs 2 or 3, wherein the DNA-binding protein or peptide is lipidated prior to binding the DNA.
5. The lipid nanoparticle of any one of paragraphs 2 to 4, wherein the DNA-binding protein or peptide is lipidated with a lipid moiety selected from the group consisting of a fatty acid, an isoprenoid and combinations thereof.
6. The lipid nanoparticle of paragraph 5, wherein the fatty acid is a triglyceride, a phospholipid or a cholesteryl ester.
7. The lipid nanoparticle of any one of paragraphs 2 to 6, wherein the DNA-binding protein or peptide is lipidated on a nucleophilic side chain, at the N-terminal end and/or at the C-terminal end.
8. The lipid nanoparticle of paragraph 7, wherein the nucleophilic side chain is a cysteine, a serine, a threonine, a tyrosine and/or a lysine amino acid residue.
9. The lipid nanoparticle of any one of paragraphs 2 to 8, wherein the DNA-binding protein or peptide is lipidated by palmitoylation, myristoylation, fatty-acylation, esterification, prenylation, or combinations thereof.
10. The lipid nanoparticle of paragraph 9, wherein the DNA-binding protein or peptide is lipidated by N-terminal cysteine palmitoylation, N-terminal glycine myristoylation, lysine N-acylation, C-terminal cholesterol esterification, cysteine prenylation, serine O-acylation or combinations thereof.
11. The lipid nanoparticle of paragraph 9 or 10, wherein the prenylation is farnesylation or geranylgeranylation.
12. The lipid nanoparticle of any one of paragraphs 5 to 11, wherein the lipid moiety is linked to the DNA-binding protein or peptide by a thioether bond, an ester bond, a thioester bond and/or an amide bond.
13. The lipid nanoparticle of any one of paragraphs 2 to 12, wherein the DNA-binding protein or peptide is lipidated using chemical or enzymatic lipidation.
14. The lipid nanoparticle of paragraph 13, wherein the DNA-binding protein or peptide is lipidated using chemical lipidation selected from the group consisting of chemical ligation, click chemistry, expressed protein ligation and combinations thereof.
15. The lipid nanoparticle of paragraph 13, wherein the DNA-binding protein or peptide is lipidated using enzymatic lipidation selected from the group consisting of Sortase-A mediated lipidation, transglutaminase mediated lipidation and combinations thereof.
16. The lipid nanoparticle of paragraph 15, wherein the enzymatic lipidation is performed in vivo or in vitro.
17. The lipid nanoparticle of any one of paragraphs 1 to 16, wherein the DNA-binding protein or peptide encapsulates the DNA.
18. The lipid nanoparticle of any one of paragraphs 1 to 17, wherein the DNA-binding protein or peptide binds directly to the DNA.
19. The lipid nanoparticle of any one of paragraphs 1 to 18, wherein the DNA-binding protein or peptide:
   a) reduces toxicity of the lipid nanoparticle, and/or
   b) stabilizes the DNA, and/or
   c) facilitates importation through the nuclear membrane, and/or
   d) protects the DNA from degradation, and/or
   e) facilitates nucleation of the lipid nanoparticle,
   f) increases immunogenicity of the DNA, and/or
   g) inhibits induction of signalling by one or more Toll-like Receptors.
20. The lipid nanoparticle of any one of paragraphs 1 to 19, wherein the DNA-binding protein or peptide is a viral or non-viral DNA-binding protein or peptide.
21. The lipid nanoparticle of paragraph 20, wherein the viral DNA-binding protein is from a class I, class II, and/or class VII virus.
22. The lipid nanoparticle of paragraph 21, wherein the viral DNA binding protein or peptide is virus selected from the group consisting of an adenovirus, a herpesvirus, a poxvirus, an adeno-associated virus, a geminivirus, a bacteriophage, a parvovirus, a heparnavirus, a hepadnavirus, a circoviridae virus, and a papovaviridae virus.
23. The lipid nanoparticle of paragraph 22, wherein the viral DNA-binding protein or peptide is a nucleoprotein, a non-structural protein, a matrix protein and/or a nucleocapsid protein.
24. The lipid nanoparticle of paragraph 20, wherein the non-viral DNA binding protein or peptide is derived from a cellular protein associated with cell growth, cell signalling and/or anti-viral pathways.
25. The lipid nanoparticle of paragraph 24, wherein the cellular protein is selected from the group consisting of a TAR DNA binding protein (TRBP), Y-box binding protein, Z-DNA Binding Protein and combinations thereof.
26. The lipid nanoparticle of any one of paragraphs 1 to 25, wherein the lipid nanoparticle additionally comprises a PEG-lipid, a structural lipid, an ionisable lipid and/or a neutral lipid.
27. The lipid nanoparticle of any one of paragraphs 1 to 26, wherein the lipid nanoparticle does not comprise a cationic lipid.
28. The lipid nanoparticle of any one of paragraphs 1 to 27, wherein the DNA is non-linear DNA.
29. The lipid nanoparticle of paragraph 28, wherein the DNA is a plasmid.
30. A composition comprising the lipid nanoparticle of any one of paragraphs 1 to 29.
31. A pharmaceutical composition comprising the lipid nanoparticle of any one of paragraphs 1 to 29 or the composition of paragraph 30 and a pharmaceutically acceptable carrier.
32. The lipid nanoparticle of any one of paragraphs 1 to 29, composition of paragraph or the pharmaceutical composition of paragraph 31 for use in therapy.
33. A method for delivering DNA to the nucleus of a cell, the method comprising contacting the cell with the lipid nanoparticle of any one of paragraphs 1 to 29.
34. A method for delivering DNA to a cell in a subject, the method comprising administering to the subject the lipid nanoparticle of any one of paragraphs 1 to 29, composition of paragraph 30 or the pharmaceutical composition of paragraph 31.

### EXAMPLES

### Example 1: DNA-Free NP Binds DNA

To assess whether DNA-free nucleoprotein (NP) protects DNA, NP was combined with nanoluciferase and samples were analysed by Agarose Gel electrophoresis following heating to 40° C. or incubation at room temperature.

The samples were also analysed using SDS-PAGE, electrophoretic mobility shift assay (EMSA) and size exclusion-high-performance liquid chromatography (HPLC-SEC). Samples included four nucleoproteins derived from influenza A, beak and feather disease virus (BFDV), Human Papillomavirus (HPV), and Hepatitis B (HBV).

The HPLC-SEC was run on a Superdex 200 Increase column at 4 mL/min in PBS.

The EMSA was run using a native PAGE gel with a binding buffer of TEA (40 mM Tris, 1 mM EDTA) with 10% glycerol, 0.1 mM DTT, 100 mM KCl, 0.1 mg/mL BSA (no Mg²⁺) at 180V for 40 min. 33 ng of DNA complexed with varying amount of DNA in 20 uL total.

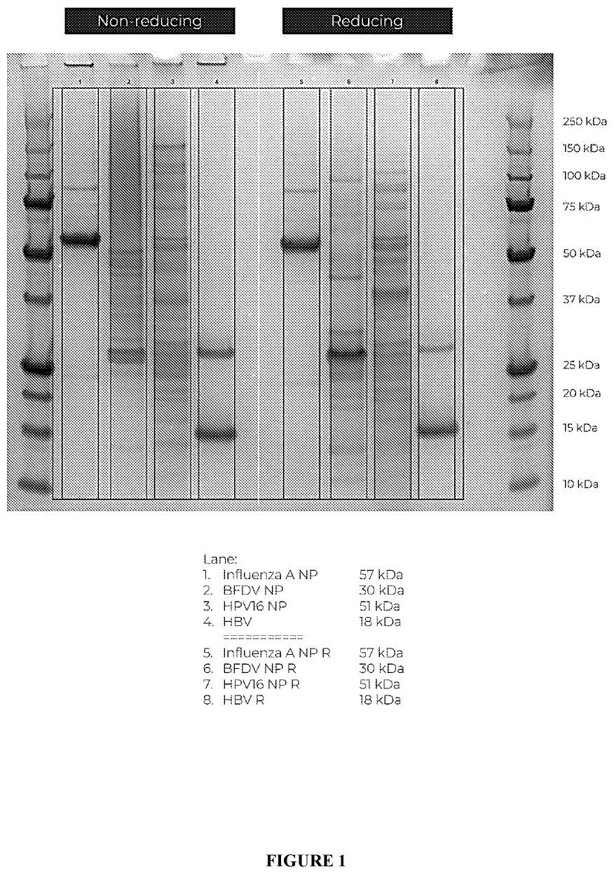

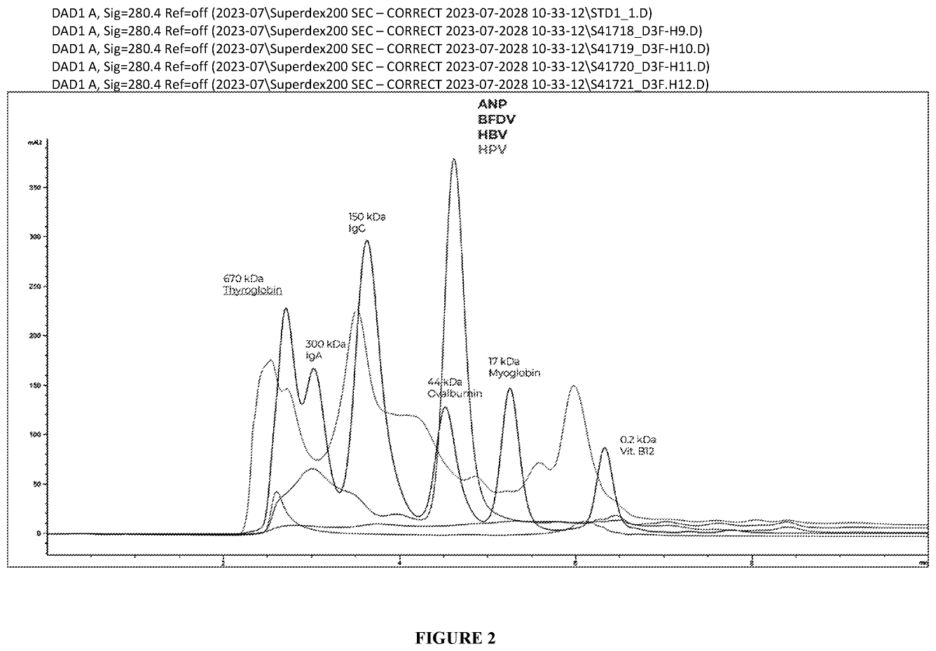

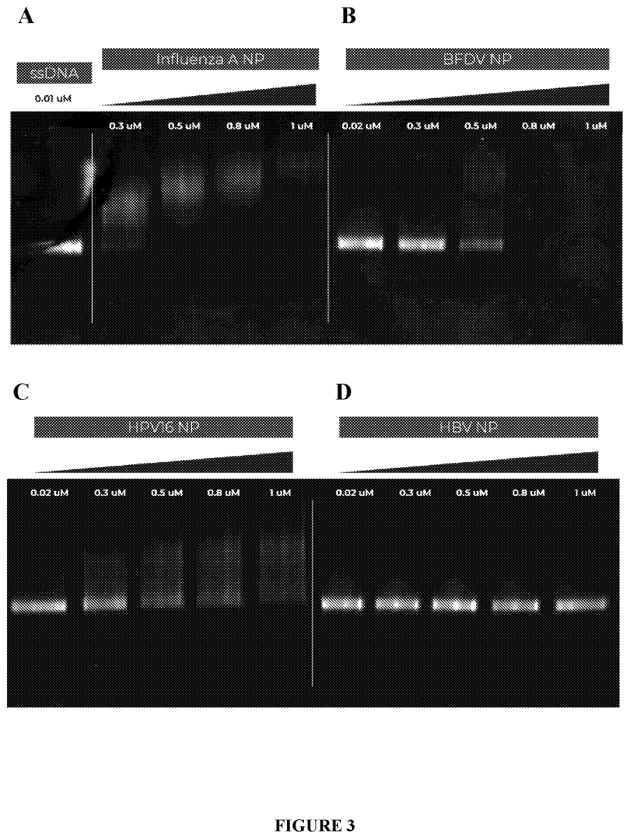

As shown in FIGS. 1 and 3 and Table 2, influenza A binds to ssDNA well. FIG. 2 illustrates that influenza A elutes well.

As shown in FIG. 1, BFDV has some impurities, and appears to oligomerise, which can be mostly recovered under reducing conditions. FIG. 2 demonstrates poor elution of BFDV in non-reducing condition. FIG. 3 and Table 2 illustrate that BFDV binds ssDNA but only above 500 nM (DNA at 10 nM) or ˜10:1 w/w ratio of protein to DNA—reducing buffer, without Mg²⁺.

As shown in FIG. 1, HPV16 has many impurities, either oligomerises or degrades, and does not seem to be recoverable with reducing conditions. The data in FIG. 2 reflects this. However, as illustrated in FIG. 3 and Table 2 HPV16 binds ssDNA in gel shift similarly to BFDV.

As shown in FIG. 1, HBV appears to dimerise, which can be partially recovered under reducing conditions. FIG. 2 demonstrates aggregation under non-reduction conditions. FIG. 3 and Table 2 illustrates that HBV does not bind to ssDNA at a low MW.

TABLE 2
Mass ratio of protein:DNA

| | | | | |
|---|---|---|---|---|
| Protein Conc. | ANP | BFDV | HPV16 | HBV |

| | | | | | |
|---|---|---|---|---|---|
| 0.02 | uM | | 0.4 | 0.6 | 0.2 |
| 0.3 | uM | 9.2 | 4.8 | 8.4 | 2.9 |
| 0.5 | uM | 17.7 | 9.3 | 16.1 | 5.5 |
| 0.8 | uM | 26.2 | 13.8 | 23.8 | 8.2 |
| 1 | uM | 34.8 | 18.3 | 31.5 | 10.8 |

### Example 2: DNA-Free NP Protects DNA from Degradation

To assess whether DNA-free NP protects DNA, the NP:DNA and DNA alone is assessed in a DNase assay. Briefly, NP:DNA or DNA is treated with DNase and incubated for 5-10 minutes at 30° C. Samples are further treated with or without 1 thermolabile proteinase K (PK). The reaction is incubated at 37° C. for 15-30 minutes, followed by incubation at 60° C. for 10-20 minutes to inactivate the PK. 1-2 μl DNase is added if required. The level of DNA present is assessed in treated and untreated samples using real time PCR.

### Example 3: DNA-Free NP Protects DNA from Degradation at 4° C., 24° C. And 37° C.

To assess whether the ability of NP to protect nanoluciferase DNA (nLuc DNA) is temperature dependent, NP:nLuc DNA and nLuc DNA are incubated at 4° C., 24° C. and 37° C. for up to 96 hours and the amount of luciferase produced is assessed by measuring luminescence in RLU.

### Example 4: DNA-Free NP Protects TLR Induction

To assess whether the presence of NP inhibits DNA induction of dsDNA or ssDNA, TLR-3, TLR-7, TLR-8 and/or TLR-9 induction is assessed using a TLR induction NfKB reporter assay. In this assay, NfKB is operationally linked to a secretary alkaline phosphatase (SEAP). DNA is introduced into either cell type (TLR-3 conditionally transduced, TLR-7 conditionally transduced, TLR-8 conditionally transduced or TLR-9 conditionally transduced). Binding of the TLR receptor induces NfKB activation and in turn SEAP. The SEAP level can be determined by a chemical reaction and calorific read out.

### Example 5: DNA-Free NP Conjugation with Maleimide-DSPE Lipid

NP (0.85 mg/ml) is conjugated with maleimide-DSPE (1,2-Distearoyl-sn-glycero-3-phosphorylethanolamine; 8 mM) by incubating NP with DSPE in the presence of 10% ethanol. The labelled and unlabelled NP are run on a non-reducing gel to assess molecular weight of the protein and confirm conjugation of the protein with a molecular weight shift of the NP. In addition, DSPE forms a suspension (i.e., liposomes) so the composition is centrifuged and the supernatant assessed.

### Example 6: Preparation of DNA-Binding Proteins and Peptides

Cellular and viral protein sequences are reviewed to identify protein domains and peptide sequences that have the potential to bind DNA. Sequences from cellular proteins correlate to those proteins associated with cell growth, cell signalling and/or antiviral pathways whereas sequences from viral proteins are derived from non-structural and nuclear proteins.

Proteins are designed from sequences derived from e.g., hepatitis B virus (HBV). Peptides are designed from sequences derived from cellular proteins including, but not limited to, TAR DNA binding protein (TDBP), Y-box binding protein, and Z-DNA Binding Protein.

### Example 7: NP Binding to DNA at Different Ratios

To further assess the four nucleoproteins derived from influenza A, BFDV, HPV, and HBV, HPLC sizing, SDS-PAGE and EMSA are conducted. The four nucleoproteins are mixed at 5 different ratios with ssDNA (molar ratio of protein:DNA ranging from 0.5:1 to 100:1).

16 ng of ssDNA are used per complexing step, all loaded on Native PAGE gel using a TEA buffer (40 mM Tris, 1 mM EDTA) with 10% glycerol, 0.1 mM DTT, 100 mM KCl and 0.1 mg/mL BSA. A buffer comprising 100 mM Tris-HCl, pH 8, 300 mM KCl, 25 mM MgCl₂, 20% glycerol, 500 g/ml BSA is also used for the BFDV NP.

0.5 ug of ssDNA is electroporated into 5E5 cells using 10 ul Neon tips per condition. For example, 3:1 or 9:1 molar ratio of protein to ssDNA is used. Controls are ssDNA electroporated, mock electroporation, and proteins electroporated alone at the highest dose.

### Example 8: Optimal Protein to ssDNA Ratio for Binding and Nuclear Delivery

To assess the optimal protein:ssDNA ratio for binding and nuclear delivery four nucleoproteins derived from influenza A, BFDV, HPV, and HBV are assessed. These NPs are mixed with ssDNA of 528 nt in length containing no viral sequences to form deoxyribonucleoproteins (DNPs). The optimal ratio of protein to ssDNA is determined within the range of 2-100 moles/moles based on an electrophoretic mobility shift assay.

The identified optimal ratio is used to form nucleoprotein-ssDNA complexes prior to electroporation into K562 traffic light reporter cells. Improved levels of homology-directed repair (HDR) insertion is determined based on insertion and NHEJ-induced indels.

### Example 9: Encapsulation of ssDNA:Nucleoprotein Complexes into Lipid Nanoparticles

To assess the encapsulation of ssDNA:NP complexes into lipid nanoparticles the four nucleoproteins derived from influenza A, BFDV, HPV, and HBV are assessed. These NPs are mixed with ssDNA of 528 nt in length containing no viral sequences, using the optimal ratio identified in Example 8 to form DNPs. The DNPs are encapsulated using a standard microfluidic mixer within lipid nanoparticles (LNPs), formulated with a mixture of ionisable lipid/cationic lipid helper lipid (DSPC), cholesterol, and DSPE-PEG lipids with ratios ranging from 10-50:10-30:10-50:0.5-2.5 respectively. The LNPs are purified by standard processes.

The degree of encapsulation is determined via BCA and OliGreen ssDNA assays. The size and polydispersity are measured via dynamic light scattering (DLS). The zeta potential is measured by electrophoretic light scattering.

### Example 10: Improved Homology-Directed Repair (HDR) Via DNP-LNPs

To assess HDR, the optimal DNP-LNPs formulations from Example 9 are used for encapsulation. K562 traffic light reporter cells are transfected with increasing doses of DNP-encapsulated LNPs alongside LNPs containing mRNA coding for Cas9 and sgRNA. A control without nucleoprotein, only ssDNA, is also included alongside the DNP-LNPs. At day 5 post transfection, levels of HDR and non-homologous end joining (NHEJ) are determined via fluorescence-activated cell sorting (FACS). Optimal doses and formulations for improved HDR due to the nucleoproteins are identified.

### Example 11: Improved HDR in CD34+ Haematopoietic Stem and Progenitor Cells

To assess HDR in CD34+ cells the optimal DNP-LNPs formulations from Example 9 are used. DNP HDR templates and ssDNA alone are assessed for HDR efficiency in CD34+ haematopoietic stem and progenitor cells ex vivo from three donors.

A guide targeting the globin locus is used along with a short ssDNA HDR template including 100 nt homology arms. Addition of ApoE3 to the culture media is used to ensure adequate LNP transfection of cells. Readout is done via Sanger sequencing 3 days post transfection.

1. A lipid nanoparticle for delivery of DNA, the lipid nanoparticle comprising therein a DNA-binding protein or peptide bound to the DNA.

2. The lipid nanoparticle of claim 1, wherein the DNA-binding protein or peptide is a lipidated DNA-binding protein or peptide.

3. A lipid nanoparticle for delivery of DNA, the lipid nanoparticle comprising therein a lipidated DNA-binding protein or peptide bound to the DNA.

4. The lipid nanoparticle of claim 2, wherein the DNA-binding protein or peptide is lipidated;
a) prior to binding the DNA;
b) with a lipid moiety selected from the group consisting of a fatty acid, an isoprenoid and combinations thereof;
c) on a nucleophilic side chain at the N-terminal end and/or at the C-terminal end,
d) by palmitoylation, myristoylation, fatty-acylation, esterification, prenylation, or combinations thereof;
e) by N-terminal cysteine palmitoylation, N-terminal glycine myristoylation, lysine N-acylation, C-terminal cholesterol esterification, cysteine prenylation, serine O-acylation or combinations thereof;
f) using chemical or enzymatic lipidation;
g) using chemical lipidation selected from the group consisting of chemical ligation, click chemistry, expressed protein ligation and combinations thereof; and/or
h) using enzymatic lipidation selected from the group consisting of Sortase-A mediated lipidation, transglutaminase mediated lipidation and combinations thereof.

5. (canceled)

6. The lipid nanoparticle of claim 4, wherein;
i) the fatty acid is a triglyceride, a phospholipid or a cholesteryl ester;
ii) the nucleophilic side chain is a cysteine, a serine, a threonine, a tyrosine and/or a lysine amino acid residue;
iii) the prenylation is farnesylation or geranylgeranylation;
iv) the lipid moiety is linked to the DNA-binding protein or peptide by a thioether bond, an ester bond, a thioester bond and/or an amide bond; and/or
v) the enzymatic lipidation is performed in vivo or in vitro.

7-16. (canceled)

17. The lipid nanoparticle of claim 1, wherein the DNA-binding protein or peptide encapsulates the DNA.

18. The lipid nanoparticle of claim 1, wherein the DNA-binding protein or peptide binds directly to the DNA.

19. The lipid nanoparticle of claim 1, wherein the DNA-binding protein or peptide:
a) reduces toxicity of the lipid nanoparticle, and/or;
b) stabilizes the DNA, and/or;
c) facilitates importation through the nuclear membrane, and/or;
d) protects the DNA from degradation, and/or;
e) facilitates nucleation of the lipid nanoparticle;
f) increases immunogenicity of the DNA; and/or
g) inhibits induction of signalling by one or more Toll-like Receptors.

20. The lipid nanoparticle of claim 1, wherein the DNA-binding protein or peptide is a viral or non-viral DNA-binding protein or peptide.

21. The lipid nanoparticle of claim 20, wherein the viral DNA-binding protein is from a class I, class II, or class VII virus.

22. The lipid nanoparticle of claim 20, wherein the viral DNA binding protein or peptide is a virus selected from the group consisting of an adenovirus, a herpesvirus, a poxvirus, an adeno-associated virus, a geminivirus, a bacteriophage, a parvovirus, a heparnavirus, a hepadnavirus, a circoviridae virus, and a papovaviridae virus.

23. The lipid nanoparticle of claim 20, wherein the viral DNA-binding protein or peptide is a nucleoprotein, a non-structural protein, a matrix protein and/or a nucleocapsid protein.

24. The lipid nanoparticle of claim 20, wherein the non-viral DNA binding protein or peptide is derived from a cellular protein associated with cell growth, cell signaling and/or anti-viral pathways.

25. The lipid nanoparticle of claim 24, wherein the cellular protein is selected from the group consisting of a TAR DNA binding protein (TRBP), Y-box binding protein, Z-DNA Binding Protein and combinations thereof.

26. The lipid nanoparticle of claim 1, wherein the lipid nanoparticle additionally comprises a PEG-lipid, a structural lipid, an ionisable lipid and/or a neutral lipid.

27. The lipid nanoparticle of claim 1, wherein the lipid nanoparticle does not comprise a cationic lipid.

28. The lipid nanoparticle of claim 1, wherein the DNA is non-linear DNA.

29. The lipid nanoparticle of claim 28, wherein the DNA is a plasmid.

30. (canceled)

31. A pharmaceutical composition comprising a lipid nanoparticle for delivery of DNA, the lipid nanoparticle comprising therein a DNA-binding protein or peptide bound to the DNA and a pharmaceutically acceptable carrier.

32. The pharmaceutical composition of claim 31 for use in therapy.

33-34. (canceled)
